(19) United States
(12) Plant Patent Application Publication
(10) Pub. No.: US 2017/0099764 P1
(43) Pub. Date: Apr. 6, 2017

(54) Cotyledon hybrid named 'Mint Truffles'

(50) ;

(71) Applicant: Renee O'Connell, Escondido, CA (US)

(72) Inventor: Renee O'Connell, Escondido, CA (US)

(21) Appl. No.: 14/756,707

(22) Filed: Oct. 1, 2015

(51) Int. Cl. A01H 5/00

(52) U.S. Cl. PLT/373

(57) ABSTRACT

A new and distinct Cotyledon cultivar named ‘Mint Truffles’ is disclosed, characterized by a compact morphology, contrasting silvery mint-green leaves margined in crimson. Plants freely offset at an early age, allowing plant to be produced commercially as a clustering 9cm. Plants, can also be produced in a larger pot size for the landscape. Free offset production at an early age enables faster propagation of the cultivar in the commercial nursery. Plants root quickly and grow vigorously; these characteristics, in combination with the tendency to freely offset at an early age, result in attractive clusters quickly, enhancing production intervals in the commercial environment. The new variety is a Cotyledon, part of the Crassulaceae complex that includes Aeonium, Echeveria, Graptopetalum, Pachyphytum, Sedum and others. Cotyledon is a popular genus, typically produced as container plants for the patio, or as landscape plants.

Latin name of the genus and species: Cotyledon hybrid.

Variety denomination: ‘Mint Truffles’.

### BACKGROUND OF THE INVENTION

The new cultivar, Cotyledon ‘Mint Truffles’, is a product of a planned breeding program. The new variety originated from a cross pollination of the proprietary, unpatented seed parent Cotyledon ‘MAC03’ with the pollen parent, the proprietary, unpatented pollen parent Cotyledon ‘ORB06’. The cross pollination was made during August, 2011, in Vista, Calif. at a commercial greenhouse. The new cultivar ‘Mint Truffles’ was discovered by the inventor, Renee O Connell, in March, 2012, at a commercial greenhouse in Vista, Calif.

Asexual reproduction of the new cultivar ‘Mint Truffles’ was first performed in Vista, Calif., at a commercial greenhouse, by vegetative terminal cuttings in April, 2012. Cotyledon ‘Mint Truffles’ has since produced multiple generations and has shown that the unique features of this cultivar are stable and reproduced true to type.

### SUMMARY OF THE INVENTION

The cultivar ‘Mint Truffles’ has not been observed under all possible environmental conditions. The phenotype may vary somewhat with variations in environment such as temperature, day length, and light intensity, without, however, any variance in genotype.

The following traits have been repeatedly observed and are determined to be the unique characteristics of Cotyledon ‘Mint Truffles’. These characteristics in combination distinguish ‘Mint Truffles’ as a new and distinct Cotyledon cultivar:
1. Cotyledon ‘Mint Truffles’ displays a low growing morphology, in combination with silvery mint-green leaves, margined with red.
2. Cotyledon ‘Mint Truffles’ exhibits a compact growth habit, enabling production for small pot sizes, such as 9 cm, as well as for 1, 2 and 5 gallon pots for the landscape.
3. Cotyledon ‘Mint Truffles’ is freely offsetting, at an early age, forming low- growing clusters quickly.
4. Cotyledon ‘Mint Truffles’ is easily and rapidly propagated due to fast rooting, vigorous growth, and prolific offsetting, enhancing production times in the commercial nursery.
5. Cotyledon ‘Mint Truffles’, when planted out in the landscape, continues to have a compact growth; rather than growing tall and lanky, the new cultivar ‘Mint Truffles’ spreads laterally.

### PARENTAL COMPARISON

Plants of the new cultivar ‘Mint Truffles’ can be compared to plants of the unpatented, proprietary Cotyledon ‘MAC03’, and are similar in most horticultural characteristics. However, plants of the new cultivar ‘Mint Truffles’ display a more compact morphology, not exhibited by Cotyledon ‘MAC03’. The new cultivar ‘Mint Truffles’ is freely offsetting at an early age, in contrast to ‘MAC03’, which produces few offsets. In addition, the new cultivar ‘Mint Truffles’ exhibits smaller mint-green leaves with a red margin, in comparison to the larger, lime-green leaves produced by the seed parent Cotyledon ‘MAC03’. The new cultivar ‘Mint Truffles’ produces compact plants with many offsets, and grows laterally to form a cluster, in contrast to the seed parent Cotyledon ‘MAC03’, which tends to become tall and leggy with sparse branching.

Plants of the new cultivar ‘Mint Truffles’ can be compared to plants of pollen parent Cotyledon ‘ORB06’, and are similar in most horticultural characteristics. However, plants of the new cultivar ‘Mint Truffles’ exhibit early and free offsetting, in contrast to the sparse offsetting produced by the seed parent by Cotyledon ‘ORB06’. Further, the new cultivar ‘Mint Truffles’ exhibits smaller mint-green leaves with bright red margins, in contrast to the grayish leaves produced by the pollen parent Cotyledon ‘ORB06’. Additionally, the new cultivar ‘Mint Truffles’ can be produced as a 9 cm pot size due to its compact growth and early offsetting, whereas the pollen parent Cotyledon ‘ORB06’ does not make an attractive 9 cm as the leaves and branches are too large, and do not convey the concept of the “small cluster” conveyed by the new cultivar ‘Mint Truffles’.

### COMMERCIAL COMPARISON

The new cultivar ‘Mint Truffles’ can be compared to the unpatented commercial variety Cotyledon orbiculata v. orbiculata. Plants of the Cotyledon orbiculata v. orbiculata are similar to plants of the new cultivar ‘Mint Truffles’ in most horticultural characteristics. However, the new cultivar ‘Mint Truffles’ exhibits a compact form, in contrast to the taller, lanky form exhibited by Cotyledon orbiculata v. orbiculata. In addition, ‘Mint Truffles’ forms smaller leaves of mint-green, margined with a contrasting bright red, whereas Cotyledon orbiculata v. orbiculata forms larger, non-uniform gray to gray white leaves. The new cultivar ‘Mint Truffles’ is freely offsetting from an early age, forming attractive clusters, whereas Cotyledon orbiculata v. orbiculata has a upright, lanky, lax growth habit, and does not produce offsets until much older. For this reason, the new cultivar, ‘Mint Truffles’ due to its quick rooting, freely offsetting and compact growth habit, produces a morphologically aesthetic cluster, thereby enhancing production time in a commercial nursery, in contrast to Cotyledon orbiculata v. orbiculata.

The new cultivar ‘Mint Truffles’ can be compared to the unpatented commercial variety Cotyledon orbiculata v. ‘Macrantha’. Plants of Cotyledon orbiculata v. ‘Macrantha are similar to plants of the new cultivar ‘Mint Truffles’ in most horticultural characteristics. However, the cultivar ‘Mint Truffles’ exhibits a more compact growth form, growing laterally, rather than vertically, as compared to the lanky, upright growth habit of Cotyledon orbiculata ‘Macrantha’. In addition, the new cultivar ‘Mint Truffles’ is freely offsetting from an early age, enhancing the propagation rate, and producing a compact, aesthetically attractive cluster, as compared with Cotyledon orbiculata ‘Macrantha’, which produces offsets sparingly and at an older age.

### BRIEF DESCRIPTION OF THE PHOTOGRAPHS

The photographs were taken using conventional techniques and equipment. While the colors in these photographs may display variances of color as compared to the living cultivar, due to LRV (light reflectance value), they are as accurate as possible using conventional photographic techniques. Colors in the photographs may appear to differ slightly from the color values cited in the botanical description, which accurately describe the colors of the new Cotyledon plant. All photographs provided by the breeder.

FIG. 1 illustrates in full color a plant typical of Cotyledon ‘Mint Truffles’ grown in full sun (approximately 9000 foot candles) in Vista, Calif.

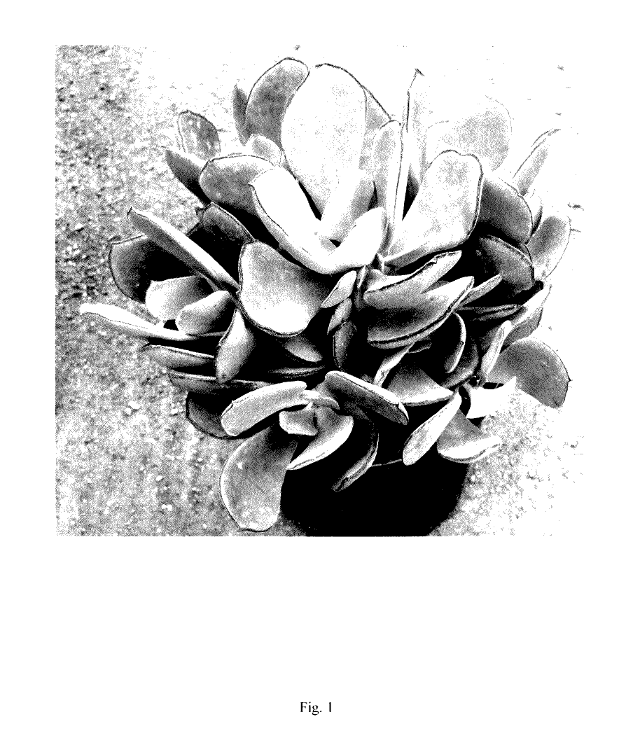

FIG. 2 illustrates in full color a plant typical of Cotyledon ‘Mint Truffles’ grown in a greenhouse (approximately 3500 foot candles) in Vista, Calif.

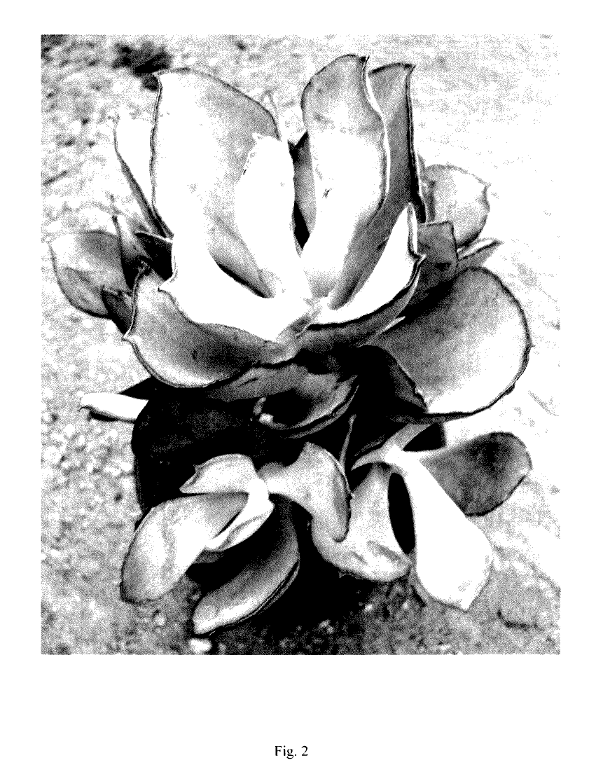

FIG. 3 illustrates in full color a plant typical of Cotyledon ‘Mint Truffles’ grown in a greenhouse (approximately 3500 foot candles) in Vista, Calif. (9 cm pot)

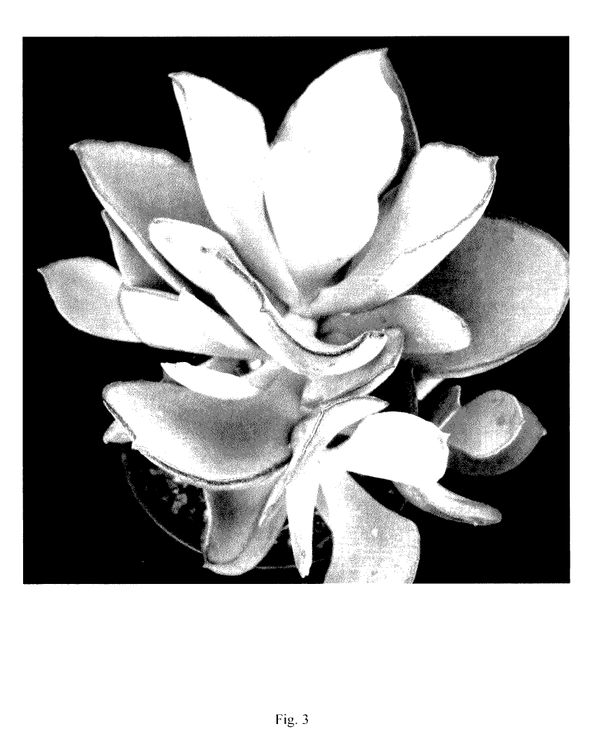

FIG. 4 illustrates in full color a close-up of a branch typical of Cotyledon ‘Mint Truffles’ grown in full sun

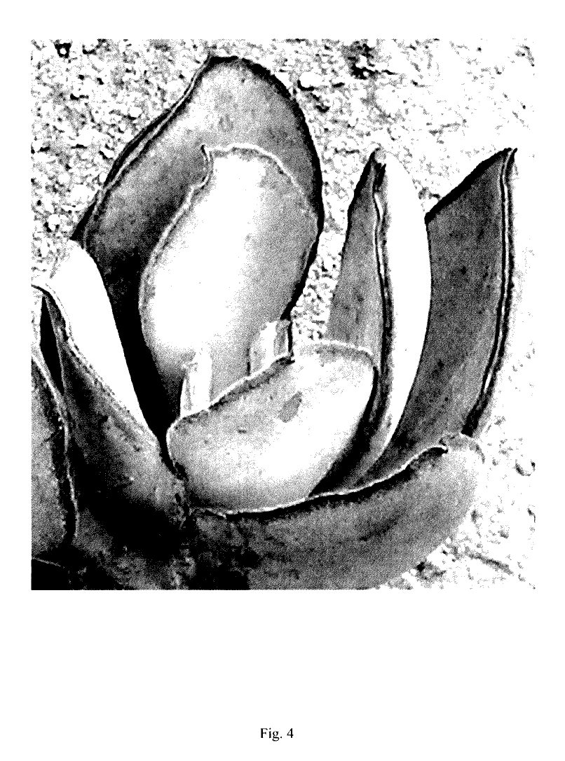

### DETAILED BOTANICAL DESCRIPTION

In the following description, color references are made to the Panton Process Color System Guide, except where general terms of ordinary dictionary significance are used. The following observations and measurements describe ‘Mint Truffles’ plants grown in full sun in Vista, Calif. Temperatures ranged from −1° C. to 29° C. night and day. No artificial light, photoperiodic treatments or chemical treatments were given to the plants. Natural light conditions were approximately 9000 fc of light. Measurements and numerical values represent averages of typical plant types.
Botanical classification: Cotyledon hybrid ‘Mint Truffles’.
Age of the plant described: Approximately 3 months from a cutting.
Propagation:
   Time to initiate roots.—About 11 days at approximately 24° C.
   Root description.—Fibrous.
   Propagation method.—Terminal vegetative cuttings.
Plant:
   Growth habit.—Upright, subshrub branching from base, and continuing to produce offsets between the leaves to form bushy clusters.
   Age of plant described.—Approximately 3 months from a cutting.
   Container size.—15.24 cm.
   Height.—Approximately 24 cm to top of highest leaf. Approximately_N/A_cm to top of highest inflorescence. Unknown as plant has been undergoing propagation, and flowering has not yet been observed.
   Plant spread.—Approximately 32 cm.
   Growth rate.—Moderately fast.
   Branching characteristics.—Freely offsetting. A 9 cm pot size had 8 branches.
Foliage:
   Leaf.—Arrangement — Decussate. Average length — Approximately 8 cm. Longest length — Approximately 11.3 cm. Widest width — Approximately 1.35 cm. Width at base — 0.7 cm. Shape of blade — Oblanceolate. Apex — Obtuse, mucronate. Base — Semicircular. Margin — Entire. Texture of top surface — Glabrous. Texture of bottom surface — Glabrous. Quantity of leaves per plant — Approximately 105, from 11 branches. Young foliage upper side — Near S 289-6 Pantone & S 289-5 Pantone. Young foliage, upper side, apical margin — Near S 99-3 Pantone. Young foliage upper side, near base — Near S 284-6 Pantone. Young foliage, under side, towards apex — Near S 289-3 Pantone. Young foliage, under side, near base — Near S 284-8 Pantone. Mature foliage upper side — Near S 289-5 Pantone. Mature foliage, under side — Near S 289-4 Pantone. Mature foliage, upper side, apical margin: — Near S 319-1 Pantone. Mature foliage, upper side (9cm grown in greenhouse, 3500 fc): — Near S 127-8 Pantone. Mature foliage, upper side, margin (9cm grown in greenhouse, 3500 fc): — Near S 137-2 Pantone.
   Venation.—There is no visual appearance of venation.
Flower: Flowering not observed to date.
Other characteristics: Fruits and seeds not observed to date.
   Temperature tolerance.—Tolerates temperatures from approximately −2° C. to 40° C.
   Disease/pest resistance.—Cotyledon ‘Mint Truffles’ displays the same disease and pest resistance of any other comparable Cotyledon.
   Drought tolerance.—Tolerates at least 4 weeks of high temperatures without supplemental water, showing no serious damage to plant.

What is claimed is:

1. A new and distinct cultivar of Cotyledon plant named ‘Mint Truffles’ as herein illustrated and described.
